(19) United States
(12) Patent Application Publication
(10) Pub. No.: US 2007/0148205 A1
(43) Pub. Date: Jun. 28, 2007

(54) Prevention of Arterial Restenosis with Active Vitamin D Compounds

(76) Inventors: Martha J. Whitehouse, San Francisco, CA (US); Bradford S. Goodwin, San Mateo, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/614,803

(22) Filed: Dec. 21, 2006

Related U.S. Application Data
(63) Continuation of application No. 10/589,435, filed on Aug. 15, 2006.
(60) Provisional application No. 60/569,241, filed on May 10, 2004.

(51) Int. Cl. A61K 31/59; A61F 2/02

(52) U.S. Cl. 424/423; 514/167

(57) ABSTRACT

The present invention relates to a method for preventing, treating, or ameliorating arterial restenosis after angioplasty in an animal by administering to the animal active vitamin D compounds. The invention further relates to a method for preventing, treating, or ameliorating restenosis after angioplasty in an animal by administering to the animal active vitamin D compounds in combination with other therapeutic agents. A further aspect of the invention is a method for preventing, treating, or ameliorating stenosis within and/or around an arterial bypass graft in an animal comprising administering to the animal an active vitamin D compound.

### BACKGROUND OF THE INVENTION

1. Field of the Invention

The present invention relates to a method for preventing, treating, or ameliorating arterial restenosis after angioplasty in an animal by administering to the animal active vitamin D compounds. The invention further relates to a method for preventing, treating, or ameliorating restenosis after angioplasty in an animal by administering to the animal active vitamin D compounds in combination with other therapeutic agents. A further aspect of the invention is a method for preventing, treating, or ameliorating stenosis within and/or around an arterial bypass graft in an animal comprising administering to the animal an active vitamin D compound.

2. Related Art

Atherosclerosis is one of the major causes of cardiovascular disease. Treatment of atherosclerotic lesions by angioplasty has become increasing popular due to the lower expense and time of recovery compared to bypass surgery. (See Harrison's Principles of Internal Medicine: Part Eight, “Coronary Angioplasty and Other Therapeutic Applications of Cardiac Catheterization,” Chapter 245, pp. 1375-1379, A. S. Fauci et al., (eds.), McGraw-Hill, New York (1998)). More than 400,000 percutaneous transluminal coronary angioplasty (PTCA) procedures are performed each year in the United States, surpassing the number of bypass operations.

While the initial success rate for PTCA is high (greater than 90%), restenosis of the dilated segment occurs in 30-45 percent of patients within 6 months. This results in the need for repeated angioplasties or bypass surgery. Restenosis is due in large part to hyperproliferation of smooth muscle cells of the intimal layer of the artery in response to injury. Restenosis following angioplasty occurs not only in arteries but also in grafts used in artery bypass operations. A similar hyperproliferative response occurs in arterial bypass grafts, likely due to the injury caused by the surgery, resulting in stenosis within and/or around the graft.

Peripheral arteries are also subject to atherosclerosis, particularly in elderly men. The most common locations for atherosclerotic lesions are in the iliac, femoral, and popliteal arteries, but lesions also occur in other arteries, e.g., aorta, cerebral, carotid, pulmonary, and renal arteries. Angioplasty of occlusions in these arteries results in high initial success rates (greater than 80%), but restenosis is prevalent.

In some instances of angioplasty a tubular metal or polymer stent is inserted after the procedure to resist elastic recoil of the vessel and to provide a larger lumen, thereby lowering the incidence of restenosis to 20-30 percent of patients. The stent may be coated or impregnated with one or more drugs that inhibit cell proliferation to prevent or ameliorate restenosis within the stent (Regar et al, Br. Med. Bull. 59:22748 (2001)). However, restenosis within the stent frequently occurs.

Vitamin D is a fat soluble vitamin which is essential as a positive regulator of calcium homeostasis. (See Harrison's Principles of Internal Medicine: Part Thirteen, “Disorders of Bone and Mineral Metabolism,” Chapter 353, pp. 2214-2226, A. S. Fauci et al., (eds.), McGraw-Hill, New York (1998)). The active form of vitamin D is 1α,25-dihydroxyvitamin D₃, also known as calcitriol. Specific nuclear receptors for active vitamin D compounds have been discovered in cells from diverse organs not involved in calcium homeostasis. (Miller et al., Cancer Res. 52:515-520 (1992)). In addition to influencing calcium homeostasis, active vitamin D compounds have been implicated in osteogenesis, modulation of immune response, modulation of the process of insulin secretion by the pancreatic B cell, muscle cell flnction, and the differentiation and growth of epidermal and hematopoietic tissues.

Moreover, there have been many reports demonstrating the utility of active vitamin D compounds in the treatment of hyperproliferative diseases (e.g., cancer and psoriasis). For example, it has been shown that certain vitamin D compounds and analogs possess potent antileukemic activity by virtue of inducing the differentiation of malignant cells (specifically, leukemic cells) to non-malignant macrophages (monocytes) and are useful in the treatment of leukemia. (Suda et al., U.S. Pat. No. 4,391,802; Partridge et al., U.S. Pat. No. 4,594,340). Anti-proliferative and differentiating actions of calcitriol and other vitamin D₃ analogues have also been reported with respect to the treatment of prostate cancer. (Bishop et al., U.S. Pat. No. 5,795,882).

Active vitamin D compounds have also been implicated in the treatment of skin cancer (Chida et al., Cancer Research 45:5426-5430 (1985)), colon cancer (Disman et al., Cancer Research 47:21-25 (1987)), and lung cancer (Sato et al., Tohoku J. Exp. Med. 138:445-446 (1982)). Other reports suggesting important therapeutic uses of active vitamin D compounds are summarized in Rodriguez et al., U.S. Pat. No. 6,034,079.

Active vitamin D compounds have also been administered in combination with other pharmaceutical agents, in particular cytotoxic agents, for the treatment of hyperproliferative disease. For example, it has been shown that pretreatment of hyperproliferative cells with active vitamin D compounds followed by treatment with cytotoxic agents enhances the efficacy of the cytotoxic agents (U.S. Pat. Nos. 6,087,350 and 6,559,139).

Although the administration of active vitamin D compounds may result in substantial therapeutic benefits, the treatment of hyperproliferative diseases with such compounds is limited by the effects these compounds have on calcium metabolism. At the levels required in vivo for effective use as anti-proliferative agents, active vitamin D compounds can induce markedly elevated and potentially dangerous blood calcium levels by virtue of their inherent calcemic activity. That is, the clinical use of calcitriol and other active vitamin D compounds as anti-proliferative agents is severely limited by the risk of hypercalcemia.

A great deal of research has gone into the identification of vitamin D analogs and derivatives that maintain an anti-proliferative effect but have a decreased effect on calcium metabolism. Hundreds of compounds have been created, many with reduced hypercalcemic effects, but no compounds have been discovered that maintain anti-proliferative activity while completely eliminating the hypercalcemic effect.

It has been shown that the problem of systemic hypercalcemia can be overcome by “high dose pulse administration” (HDPA) of a sufficient dose of an active vitamin D compound such that an anti-proliferative effect is observed while avoiding the development of severe hypercalcemia. According to U.S. Pat. No. 6,521,608, the active vitamin D compound may be administered no more than every three days, for example, once a week at a dose of at least 0.12 μg/kg per day (8.4 μg in a 70 kg person). Pharmaceutical compositions used in the HDPA regimen of U.S. Pat. No. 6,521,608 comprise 5-100 μg of active vitamin D compound and may be administered in the form for oral, intravenous, intramuscular, topical, transdernal, sublingual, intranasal, intratumoral, or other preparations.

### SUMMARY OF THE INVENTION

One aspect of the present invention is a method for preventing, treating, or ameliorating arterial restenosis after angioplasty in an animal comprising administering to the animal an active vitamin D compound. In a second aspect of the invention the active vitamin D compound has a reduced hypercalcemic effect, allowing higher doses of the compound to be administered to an animal without inducing hypercalcemia. In another embodiment of the invention the active vitamin D compound is administered by HDPA so that high doses of the active vitamin D compound can be administered to an animal without inducing hypercalcemia. Another aspect of the present invention is a method for preventing, treating, or ameliorating arterial restenosis after angioplasty in an animal comprising administering to the animal an active vitamin D compound in combination with one or more therapeutic agents. In an additional aspect of the invention, a stent is placed in the artery after angioplasty to aid in the prevention, treatment, or amelioration of restenosis. A further aspect of the invention is a method for preventing, treating, or ameliorating stenosis within and/or around an arterial bypass graft in an animal comprising administering to the animal an active vitamin D compound.

In preferred embodiments of the invention, a combination of therapeutic agents is administered. In one embodiment of the invention, vitamin D administration can start prior to administration of the one or more therapeutic agents and/or continue during and beyond administration of the one or more therapeutic agents. In another embodiment of the invention, the method of administering an active vitamin D compound in combination with one or more therapeutic agents is repeated more than once.

The combination of an active vitamin D compound with one or more therapeutic agents of the present invention can have additive potency or an additive therapeutic effect. The invention also encompasses synergistic combinations where the therapeutic efficacy is greater than additive.

Preferably, such combinations also reduce or avoid unwanted or adverse effects. In certain embodiments, the combination therapies encompassed by the invention provide an improved overall therapy relative to administration of an active vitamin D compound or any therapeutic agent alone. In certain embodiments, doses of existing or experimental therapeutic agents can be reduced or administered less frequently which increases patient compliance, thereby improving therapy and reducing unwanted or adverse effects.

Further, the methods of the invention are useful not only with previously untreated patients but also useful in the treatment of patients partially or completely refractory to current standard and/or experimental therapies for prevention, treatment, or amelioration of restenosis. In a preferred embodiment, the invention provides therapeutic methods for the prevention, treatment, or amelioration of restenosis or stenosis that has been shown to be or may be refractory or non-responsive to other therapies.

### DETAILED DESCRIPTION OF THE INVENTION

One aspect of the present invention is a method for preventing, treating, or ameliorating restenosis after angioplasty in an animal comprising administering to the animal an active vitamin D compound. In a second aspect of the invention the active vitamin D compound has a reduced hypercalcemic effect, allowing higher doses of the compound to be administered to an animal without inducing hypercalcemia. A further aspect of the present invention is a method for preventing, treating, or ameliorating restenosis after angioplasty in an animal comprising administering to the animal an active vitamin D compound by HDPA so that high doses of the active vitamin D compound can be administered to an animal without inducing hypercalcemia.

Another aspect of the present invention is a method for preventing, treating, or ameliorating restenosis after angioplasty in an animal comprising administering to the animal an active vitamin D compound in combination with one or more therapeutic agents, which therapeutic agents are currently being used, have been used, or are known to be useful in the prevention, treatment, or amelioration of restenosis.

In an additional aspect of the invention, a stent is placed in the artery during or after angioplasty to aid in the prevention, treatment, or amelioration of restenosis.

A further aspect of the invention is a method for preventing, treating, or ameliorating stenosis within and/or around an arterial bypass graft in an animal comprising administering to the animal an active vitamin D compound.

The methods described herein are useful for the prevention, treatment, or amelioration of restenosis following angioplasty occurring in coronary arteries, peripheral arteries and bypass grafts. The methods are also useful for the prevention, treatment, or amelioration of stenosis occurring in bypass grafts following bypass surgery.

As used herein, the term “therapeutically effective amount” refers to that amount of the therapeutic agent sufficient to result in prevention of restenosis or stenosis, amelioration of one or more symptoms of restenosis or stenosis, or prevention of advancement of restenosis or stenosis. For example, with respect to the treatment of restenosis or stenosis, a therapeutically effective amount preferably refers to the amount of a therapeutic agent that reduces the extent of restenosis or stenosis by at least 10%, preferably at least 20%, at least 30%, at least 40%, at least 50%, at least 60%, at least 70%, at least 80%, at least 90%, or at least 100%. The extent of restenosis or stenosis can be determined by any method known in the art for visualizing blood flow, e.g., contrast angiography.

The terms “prevent, preventing, and prevention,” as used herein, are intended to refer to a decrease in the occurrence of restenosis following an angioplasty procedure or stenosis after a surgical bypass procedure. The prevention may be complete, e.g., the total absence of restenosis within six months following the angioplasty. The prevention may also be partial, such that the amount of restenosis or stenosis is less than that which would have occurred without the present invention. For example, the extent of restenosis or stenosis using the methods of the present invention may be at least 10%, preferably at least 20%, at least 30%, at least 40%, at least 50%, at least 60%, at least 70%, at least 80%, at least 90%, or at least 100% less than the amount of restenosis or stenosis that would have occurred without the present invention.

The term “restenosis,” as used herein, is intended to refer to any narrowing or constriction in an artery or artery bypass graft following an angioplasty procedure at or near that location in the vessel. Restenosis is due in large part to neo-intimal growth following the injury induced by an angioplasty procedure. The neo-intima is an accumulation of smooth muscle cells within a proteoglycan matrix that narrows the lumen of the blood vessel.

The term “stenosis,” as used herein, is intended to refer to any narrowing or constriction within and/or around an artery bypass graft following a surgical bypass procedure.

The term “therapeutic agent,” as used herein, is intended to refer to any therapeutic agent known to those of skill in the art to be effective for the prevention, treatment, or amelioration of restenosis or stenosis. Therapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices, and nucleotide sequences encoding biologically active proteins, polypeptides, or peptides), antibodies, synthetic or natural inorganic molecules, mimetic agents, and synthetic or natural organic molecules. Any agent which is known to be useful, or which has been used or is currently being used for the prevention, treatment, or amelioration of restenosis or stenosis can be used in combination with an active vitamin D compound in accordance with the invention described herein. See, e.g., Hardman et al., eds., 1996, Goodman & Gilman's The Pharmacological Basis of Therapeutics 9^{th} Ed., McGraw-Hill, New York, N.Y. for information regarding therapeutic agents which have been or a currently being used for the prevention, treatment, or amelioration of restenosis or stenosis.

Therapeutic agents useful in the methods and compositions of the invention include antineoplastic agents (e.g., actinomycin D, irinotecan, vincristine, vinblastine, methotrexate, azathioprine, fluorouracil, doxorubicin, mitomycin), vasodilators (e.g., nitrates, calcium channel blockers), anticoagulants (e.g., heparin, anti-platelet agents (e.g., aspirin, blockers of IIb/IIIa receptors), anti-thrombins (e.g., hirudin, iloprost), immunosuppressants (e.g., sirolimus, tranilast, dexamethasone, tacrolimus, everolimus, A24), collagen synthetase inhibitors (e.g., halofuginone, propyl hydroxylase, C-proteinase inhibitor, metalloproteinase inhibitor), anti-inflammatories (e.g., corticosteroids, non-steroidal anti-inflammatory drugs), 17β-estradiol, angiotensin converting enzyme inhibitors, colchicine, fibroblast growth factor antagonists, histamine antagonists, lovastatin, nitroprusside, phosphodiesterase inhibitors, prostaglandin inhibitors, suramin, serotonin blockers, thioprotease inhibitors, platelet-derived growth factor antagonists, nitric oxide, and angiopeptin. In one embodiment, the therapeutic agent is a taxane, e.g., paclitaxel or docetaxel.

Therapeutic agents can also be radioactive materials suitable for reducing cell proliferation at the site of the angioplasty or bypass surgery. Examples of suitable radioactive agents include radioisotopes, e.g., cobalt-60, cesium-137, palladium-103, phosphorus-32, yttrium-90, strontium-90, and iridium-192. Examples of the use of radioactive materials in angioplasty procedures can be found in U.S. Pat. Nos. 6,353,756, 6,192,271, 6,179,789, 6,159,142, 5,871,437, and 5,871,436.

Therapeutic agents can also be radiation treatments. External-beam radiation therapy can be directed to the site of the angioplasty or bypass procedure to reduce cell proliferation. In general, external-beam radiation therapy comprises irradiating a defined volume within a subject with a high energy beam, thereby causing the death of proliferating cells within that volume. Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652. Other radiation techniques may also be used, e.g., charged-particle radiotherapy, neutron radiotherapy, photodynamic therapy. U.S. Pat. Nos. 5,668,371, 6,400,796, 5,877,165, 5,872,107, 5,653,957, 6,283,957, 6,071,908, 6,011,563, 5,855,595, 5,716,595, and 5,707,401.

The term “stent,” as used herein, is intended to refer to any structure that is inserted into a blood vessel during or after angioplasty to prevent, treat, or ameliorate restenosis. Stents are typically made of metal or a polymer material, and come in a wide variety of structures. Examples of stents used in angioplasty procedures can be found in U.S. Pat. Nos. 6,491,718, 6,491,617, 6,353,756, 6,315,708, 6,206,915, 6,203,536, 6,192,271, 6,015,430, 5,997,563, 5,871,437, 5,695,516, 5,549,635, 5,443,500, 5,403,341, 5,334,201, 5,266,073, 5,059,211, 5,059,166, 4,990,155, 4,886,062, 4,800,882, 4,795,458, and 4,733,665. Stents can be coated or impregnated with an active vitamin D compound and/or a therapeutic agent as described above to effect local delivery of the agent to the site of the angioplasty procedure (see Regar et al., Br. Med. Bull. 59:227-48 (2001); Evers, Drug Market Dev. p. 295 (November 2003)). The coating or impregnated material may comprise a matrix that controls the release of the drugs. Examples of drug delivery stents can be found in U.S. Pat. Nos. 6,589,546, 6,335,029, 6,218,016, and 5,304,121.

The term “an active vitamin D compound in combination with one or more therapeutic agents,” as used herein, is intended to refer to the combined administration of an active vitamin D compound and one or more therapeutic agents, wherein the active vitamin D compound can be administered prior to, concurrently with, or after the administration of the therapeutic agents. The active vitamin D compound can be administered up to three months prior to or after the therapeutic agents and still be considered to be a combination treatment.

The term “active vitamin D compound,” as used herein, is intended to refer to a vitamin D compound that is biologically active when administered to a subject or contacted with cells. The biological activity of a vitamin D compound can be assessed by assays well known to one of skill in the art such as, e.g., immunoassays that measure the expression of a gene regulated by vitamin D. Vitamin D compounds exist in several forms with different levels of activity in the body. For example, a vitamin D compound may be partially activated by first undergoing hydroxylation in the liver at the carbon-25 position and then may be fully activated in the kidney by further hydroxylation at the carbon-1 position. The prototypical active vitamin D compound is 1α,25-hydroxyvitamin D₃, also known as calcitriol. A large number of other active vitamin D compounds are known and can be used in the practice of the invention. The active vitamin D compounds of the present invention include, but are not limited to, the analogs, homologs and derivatives of vitamin D compounds described in the following patents, each of which is incorporated by reference: U.S. Pat. No. 4,391,802 (1α-hydroxyvitamin D derivatives); U.S. Pat. No. 4,717,721 (1α-hydroxy derivatives with a 17 side chain greater in length than the cholesterol or ergosterol side chains); U.S. Pat. No. 4,851,401 (cyclopentano-vitamin D analogs); U.S. Pat. No. 4,866,048 and U.S. Pat. No. 5,145,846 (vitamin D₃ analogues with alkynyl, alkenyl, and alkanyl side chains); U.S. Pat. No. 5,120,722 (trihydroxycalciferol); U.S. Pat. No. 5,547,947 (fluoro-cholecalciferol compounds); U.S. Pat. No. 5,446,035 (methyl substituted vitamin D); U.S. Pat. No. 5,411,949 (23-oxa-derivatives); U.S. Pat. No. 5,237,110 (19-nor-vitamin D compounds; U.S. Pat. No. 4,857,518 (hydroxylated 24-homo-vitamin D derivatives). Particular examples include ROCALTROL (Roche Laboratories); CALCUEX injectable calcitriol; investigational drugs from Leo Pharmaceuticals including EB 1089 (24a,26a,27a-trihomo-22,24-diene-1α,25-(OH)₂-D₃, KH 1060 (20-epi-22-oxa-24a,26a,27a-trihomo-1α,25-(OH)₂-D₃), MC 1288 (1,25-(OH)₂-20-epi-D₃) and MC 903 (calcipotriol, 1α24s-(OH)₂-22-ene-26,27-dehydro-D₃); Roche Pharmaceutical drugs that include 1,25-(OH)₂-16-ene-D₃, 1,25-(OH)2-16-ene-23-yne-D₃, and 25-(OH)₂-16-ene-23-yne-D₃; Chugai Pharmaceuticals 22-oxacalcitriol (22-oxa-1α,25-(OH)₂-D₃; 1α-(OH)-D₅ from the University of Illinois; and drugs from the Institute of Medical Chemistry-Schering AG that include ZK 161422 (20-methyl-1,25-(OH)₂-D₃) and ZK 157202 (20-methyl-23-ene-1,25-(OH)₂-D₃); 1α-(OH)-D₂; 1α-(OH)-D₃ and 1α-(OH)-D₄. Additional examples include 1α,25-(OH)₂-26,27-d₆-D₃; 1α,25-(OH)₂-22-ene-D₃; 1α,25-(OH)₂-D₃; 1α,25-(OH)₂-D₂; 1α,25-(OH)₂-D₄; 1α,24,25 -(OH)₃-D₃; 1α,24,25-(OH)₃-D₂; 1α,24,25-(OH)₃-D₄; 1α-(OH)-25-FD₃; 1α-(OH)-25-FD₄; 1α-(OH)-25-FD₂; 1α,24-(OH)₂-D₄; 1α,24-(OH)₂-D₃; 1α,24-(OH)₂-D₂; 1α, 24-(OH)₂-25-FD4; 1α,24-(OH)₂-25-FD₃; 1α,24-(OH)₂-25-FD₂; 1α,25-(OH)2-26,27-F₆-22-ene-D₃; 1α,25-(OH)₂-26,27-F₆-D₃; 1α,25S-(OK)₂-26-F₃-D₃; 1α,25-(OH)₂-24-F₂-D₃; 1α,25S,26-(OH)₂-22-ene-D₃; 1α,25R,26-(OH)₂-22-ene-D₃; 1α,25-(OH)₂-D₂; 1α,25-(OH)₂-24-epi-D₃; 1α,25-(OH)₂-23-yne-D₃; 1α,25-(OH)₂-24R-F-D₃; 1α,25S,26-(OH)₂-D₃; 1α,24R-(OH)₂-25F-D₃; 1α,25 -(OH)2-26,27-F6-23-yne-D₃; 1α,25R-(OH)₂-26-F₃-D₃; 1α,25,28-(OH)₃-D₂; 1α,25-(OH)₂-16-ene-23-yne-D₃; 1α,24R,25-(OH)₃-D₃; 1α,25-(OH)₂-26,27 -F₆-23-ene-D₃; 1α,25R-(OH)₂-22-ene-26-F₃-D₃; 1α,25S-(OH)₂-22-ene-26-F₃-D₃; 1α,25R-(OH)₂-D₃-26,26,26-d₃; 1α,25S-(OH)₂-D₃-26,26,26-d₃; and 1α,25R-(OH)₂-22-ene-D₃-26,26,26-d₃. Additional examples can be found in U.S. Pat. No. 6,521,608. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

In a preferred embodiment of the invention, the active vitamin D compound has a reduced hypercalcemic effect as compared to vitamin D so that increased doses of the compound can be administered without inducing hypercalcemia in the animal. A reduced hypercalcemic effect is defined as an effect which is less than the hypercalcemic effect induced by administration of an equal dose of 1α,25-hydroxyvitamin D₃ (calcitriol). As an example, EB 1089 has a hypercalcemic effect which is 50% of the hypercalcemic effect of calcitriol. Additional active vitamin D compounds having a reduced hypercalcemic effect include Ro23-7553 and Ro24-5531 available from Hoffman LaRoche. Other examples of active vitamin D compounds having a reduced hypercalcemic effect can be found in U.S. Pat. No. 4,717,721. Determining the hypercalcemic effect of an active vitamin D compound is routine in the art and can be carried out as disclosed in Hansen et al., Curr. Pharm. Des. 6:803-828 (2000).

In one embodiment of the invention, an active vitamin D compound is administered to an animal before, during and/or after an angioplasty procedure or bypass procedure. The active vitamin D compound can be administered 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, 4 weeks, or more prior to the angioplasty or bypass procedure. The active vitamin D compound can be administered 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, 4 weeks, or more after the angioplasty or bypass procedure and continued for up to six months. In certain embodiments the active vitamin D compound is administered before, during, and after the angioplasty procedure or bypass procedure.

In one aspect of the invention, one or more therapeutic agents are administered to an animal in addition to the active vitamin D compound. The active vitamin D compound can be administered prior to (e.g., 0.5 hours, 1 hour, 2 hours, 4 hours, 6 hours, 12 hours, 24 hours, 36 hours, 2 days, 3 days, 4 days, 5 days, 6 days, 7 days, 2 weeks, 3 weeks, 4 weeks or more), concurrently with, or after (e.g., 0.5 hours, 1 hour, 2 hours, 4 hours, 6 hours, 12 hours, 24 hours, 36 hours, 2 days, 3 days, 4 days, 5 days, 6 days, 7 days, 2 weeks, 3 weeks, 4 weeks or more) the administration of one or more therapeutic agents.

In certain embodiments, the method of administering an active vitamin D compound in combination with one or more therapeutic agents may be repeated at least once. The method may be repeated as many times as necessary to achieve or maintain a therapeutic response, e.g., from one to about ten times. With each repetition of the method the active vitamin D compound and the one or more therapeutic agents may be the same or different from that used in the previous repetition. Additionally, the time period of administration of the active vitamin D compound and the manner in which it is administered (i.e., daily or HDPA) can vary from repetition to repetition.

In some embodiments of the invention, a stent is introduced into the artery during or after the angioplasty procedure. Any stent known to one of skill in the art to be useful to prevent, treat, or ameliorate restenosis can be used in the present invention. In certain embodiments, the stent may be coated or impregnated with an active vitamin D compound, with one or more therapeutic agents, or with both. The vitamin D compound and therapeutic agents are optionally contained within a matrix which is coated on or impregnated in the stent, the matrix controlling the release of the drugs.

When used, the one or more therapeutic agents are administered in doses known to one of skill in the art to prevent, treat, or ameliorate restenosis after angioplasty or stenosis after bypass surgery. The one or more therapeutic agents are administered in pharmaceutical compositions and by methods known to be effective. For example, the therapeutic agents may be administered systemically (e.g., intravenously, orally) or locally.

The active vitamin D compound is preferably administered at a dose of about 0.5 μg to about 300 μg, more preferably from about 15 μg to about 200 μg. In a specific embodiment, an effective amount of an active vitamin D compound is 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 μg or more.

In certain embodiments, an effective dose of an active vitamin D compound is between about 3 μg to about 300 μg, more preferably between about 15 μg to about 260 μg, more preferably between about 30 μg to about 240 μg, more preferably between about 50 μg to about 220 μg, more preferably between about 75 μg to about 200 μg. In certain embodiments, the methods of the invention comprise administering an active vitamin D compound in a dose of about 0.12 μg/kg bodyweight to about 3 μ/kg bodyweight. The compound may be administered by any route, including oral, intramuscular, intravenous, parenteral, rectal, nasal, topical, or transdermal.

If the active vitamin D compound is to be administered daily, the dose may be kept low, for example about 0.5 μg to about 5 μg, in order to avoid or diminish the induction of hypercalcemia. If the active vitamin D compound has a reduced hypercalcemic effect a higher daily dose may be administered without resulting in hypercalcemia, for example about 10 μg to about 20 μg or higher (up to about 50 μg to about 100 μg).

In a preferred embodiment of the invention, the active vitamin D compound is administered by IIDPA so that high doses of the active vitamin D compound can be administered without inducing hypercalcemia IEDPA refers to intermittently administering an active vitamin D compound on either a continuous intermittent dosing schedule or a non-continuous intermittent dosing schedule. High doses of active vitamin D compounds include doses greater than about 3 μg as discussed in the sections above. Therefore, in certain embodiments of the invention, the methods for the prevention, treatment, or amelioration of restenosis or stenosis encompass intermittently administering high doses of active vitamin D compounds. The frequency of the HDPA can be limited by a number of factors including, but not limited to, the pharmacokinetic parameters of the compound or formulation and the pharmacodynamic effects of the active vitamin D compound on the animal.

For example, animals having impaired renal function may require less frequent administration of the active vitamin D compound because of the decreased ability of those animals to excrete calcium.

The following is exemplary only and merely serves to illustrate that the term HDPA can encompass any discontinuous administration regimen designed by a person of skill in the art.

In one example, the active vitamin D compound can be administered not more than once every three days, every four days, every five days, every six days, every seven days, every eight days, every nine days, or every ten days. The administration can continue for one, two, three, or four weeks or one, two, or three months, or longer. Optionally, after a period of rest, the active vitamin D compound can be administered under the same or a different schedule. The period of rest can be one, two, three, or four weeks, or longer, according to the pharmacodynamic effects of the active vitamin D compound on the animal.

In another example, the active vitamin D compound can be administered once per week for three months.

In a preferred embodiment, the vitamin D compound can be administered once per week for three weeks of a four week cycle. After a one week period of rest, the active vitamin D compound can be administered under the same or different schedule.

Further examples of dosing schedules that can be used in the methods of the present invention are provided in U.S. Pat. No. 6,521,608, which is incorporated by reference in its entirety.

The above-described administration schedules are provided for illustrative purposes only and should not be considered limiting. A person of skill in the art will readily understand that all active vitamin D compounds are within the scope of the invention and that the exact dosing and schedule of administration of the active vitamin D compounds can vary due to many factors.

The amount of a therapeutically effective dose of a pharmaceutical agent in the acute or chronic management of a disease or disorder may differ depending on factors including, but not limited to, the disease or disorder treated, the specific pharmaceutical agents and the route of administration.

According to the methods of the invention, an effective dose of an active vitamin D compound is any dose of the compound effective to prevent, treat, or ameliorate restenosis or stenosis. A high dose of an active vitamin D compound can be a dose from about 3 μg to about 300 μg or any dose within this range as discussed above. The dose, dose frequency, duration, or any combination thereof, may also vary according to age, body weight, response, and the past medical history of the animal as well as the route of administration, pharmacokinetics, and pharmacodynamic effects of the pharmaceutical agents. These factors are routinely considered by one of skill in the art.

The rate of absorption and clearance of vitamin D compounds are affected by a variety of factors that are well known to persons of skill in the art. As discussed above, the pharmacokinetic properties of active vitamin D compounds limit the peak concentration of vitamin D compounds that can be obtained in the blood without inducing the onset of hypercalcemia. The rate and extent of absorption, distribution, binding or localization in tissues, biotransformation, and excretion of the active vitamin D compound can all affect the frequency at which the pharmaceutical agents can be administered.

In one embodiment of the invention, an active vitamin D compound is administered at a dose sufficient to achieve peak plasma concentrations of the active vitamin D compound of about 0.1 nM to about 25 nM. In certain embodiments, the methods of the invention comprise administering the active vitamin D compound in a dose that achieves peak plasma concentrations of 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 12.5 nM, 15 nM, 17.5 nM, 20 nM, 22.5 nM, or 25 nM or any range of concentrations therein. In other embodiments, the active vitamin D compound is administered in a dose that achieves peak plasma concentrations of the active vitamin D compound exceeding about 0.5 nM, preferably about 0.5 nM to about 25 nM, more preferably about 5 nM to about 20 nM, and even more preferably about 10 nM to about 15 nM.

In another preferred embodiment, the active vitamin D compound is administered at a dose of at least about 0.12 μg/kg bodyweight, more preferably at a dose of at least about 0.5 μg/kg bodyweight.

One of skill in the art will recognize that these standard doses are for an average sized adult of approximately 70 kg and can be adjusted for the factors routinely considered as stated above.

In certain embodiments, the methods of the invention further comprise administering a dose of an active vitamin D compound that achieves peak plasma concentrations rapidly, e.g., within four hours. In further embodiments, the methods of the invention comprise administering a dose of an active vitamin D compound that is eliminated quickly, e.g., with an elimination half-life of less than 12 hours.

While obtaining high concentrations of the active vitamin D compound is beneficial, it must be balanced with clinical safety, e.g., hypercalcemia.

Thus, in one aspect of the invention, the methods of the invention encompass HDPA of active vitamin D compounds to an animal before, during, or after angioplasty or bypass surgery and monitoring the animal for symptoms associated with hypercalcemia. Such symptoms include calcification of soft tissues (e.g., cardiac tissue), increased bone density, and hypercalcemic nephropathy. In still another embodiment, the methods of the invention encompass HDPA of an active vitamin D compound to an animal before, during, or after angioplasty or bypass surgery and monitoring the calcium plasma concentration of the animal to ensure that the calcium plasma concentration is less than about 10.2 mg/dL.

In certain embodiments, high blood levels of vitamin D compounds can be safely obtained in conjunction with reducing the transport of calcium into the blood. In one embodiment, higher active vitamin D compound concentrations are safely obtainable without the onset of hypercalcemia when administered in conjunction with a reduced calcium diet. In one example, the calcium can be trapped by an adsorbent, absorbent, ligand, chelate, or other binding moiety that cannot be transported into the blood through the small intestine. In another example, the rate of osteoclast activation can be inhibited by administering, for example, a bisphosphonate such as, e.g., zoledronate, pamidronate, or alendronate, or a corticosteroid such as, e.g., dexamethasone or prednisone, in conjunction with the active vitamin D compound.

In certain embodiments, high blood levels of active vitamin D compounds are safely obtained in conjunction with maximizing the rate of clearance of calcium. In one example, calcium excretion can be increased by ensuring adequate hydration and salt intake. In another example, diuretic therapy can be used to increase calcium excretion. μWhen the active vitamin D compound is delivered locally, e.g., as a coating on a stent, blood levels of active vitamin D compound or calcium do not need to be monitored as the localized delivery is unlikely to result in systemically detectable levels of the active vitamin D compound or to affect systemic calcium levels.

The active vitamin D compound may be administered as part of a pharmaceutical composition comprising a pharmaceutically acceptable carrier, wherein the active vitamin D compound is present in an amount which is effective to achieve its intended purpose, i.e., to have an anti-proliferative effect. The pharmaceutical composition may further comprise one or more excipients, diluents or any other components known to persons of skill in the art and germane to the methods of formulation of the present invention. The pharmaceutical composition may additionally comprise other compounds typically used as adjuncts during prevention, treatment, or amelioration of restenosis.

The term “pharmaceutical composition” as used herein is to be understood as defining compositions of which the individual components or ingredients are themselves pharmaceutically acceptable, e.g., where oral administration is foreseen, acceptable for oral use and, where topical administration is foreseen, topically acceptable.

The pharmaceutical composition can be prepared in single unit dosage forms. The dosage forms are suitable for oral, mucosal (nasal, sublingual, vaginal, buccal, rectal), parenteral (intravenous, intramuscular, intraarterial), or topical administration. Preferred dosage forms of the present invention include oral dosage forms and intravenous dosage forms.

Intravenous forms include, but are not limited to, bolus and drip injections. In preferred embodiments, the intravenous dosage forms are sterile or capable of being sterilized prior to administration to a subject since they typically bypass the subject's natural defenses against contaminants.

Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

In a preferred embodiment of the invention, the pharmaceutical compositions comprising active vitamin D compounds are emulsion pre-concentrate formulations. The compositions of the invention meet or substantially reduce the difficulties associated with active vitamin D compound therapy hitherto encountered in the art including, in particular, undesirable pharmacolinetic parameters of the compound upon administration to a patient.

According to one aspect of the present invention, a pharmaceutical composition is provided comprising (a) a lipophilic phase component, (b) one or more surfactants, (c) an active vitamin D compound; wherein said composition is an emulsion pre-concentrate, which upon dilution with water, in a water to composition ratio of about 1:1 or more of said water, forms an emulsion having an absorbance of greater than 0.3 at 400 nm. The pharmaceutical composition of the invention may further comprise a hydrophilic phase component.

In another aspect of the invention, a pharmaceutical emulsion composition is provided comprising water (or other aqueous solution) and an emulsion pre-concentrate.

The term “emulsion pre-concentrate,” as used herein, is intended to mean a system capable of providing an emulsion upon contacting with, e.g., water. The term “emulsion,” as used herein, is intended to mean a colloidal dispersion comprising water and organic components including hydrophobic (lipophilic) organic components. The term “emulsion” is intended to encompass both conventional emulsions, as understood by those skilled in the art, as well as “sub-micron droplet emulsions,” as defmed immediately below.

The term “sub-micron droplet emulsion,” as used herein is intended to mean a dispersion comprising water and organic components including hydrophobic (lipophilic) organic components, wherein the droplets or particles formed from the organic components have an average maximum dimension of less than about 1000 mn.

Sub-micron droplet emulsions are identifiable as possessing one or more of the following characteristics. They are formed spontaneously or substantially spontaneously when their components are brought into contact, that is without substantial energy supply, e.g., in the absence of heating or the use of high shear equipment or other substantial agitation. They exhibit thermodynamic stability and they are monophasic.

The particles of a sub-micron droplet emulsion may be spherical, though other structures are feasible, e.g. liquid crystals with lamellar, hexagonal or isotropic symmetries. Generally, sub-micron droplet emulsions comprise droplets or particles having a maximum dimension (e.g., average diameter) of between about 50 nm to about 1000 nm, and preferably between about 200 nm to about 300 nm.

The pharmaceutical compositions of the present invention will generally form an emulsion upon dilution with water. The emulsion will form according to the present invention upon the dilution of an emulsion pre-concentrate with water in a water to composition ratio of about 1:1 or more of said water. According to the present invention, the ratio of water to composition can be, e.g., between 1:1 and 5000:1. For example, the ratio of water to composition can be about 1:1, 2:1, 3:1, 4:1, 5:1, 10:1, 200:1, 300:1, 500:1, 1000:1, or 5000:1. The skilled artisan will be able to readily ascertain the particular ratio of water to composition that is appropriate for any given situation or circumstance.

According to the present invention, upon dilution of said emulsion pre-concentrate with water, an emulsion will form having an absorbance of greater than 0.3 at 400 nm. The absorbance at 400 nm of the emulsions formed upon 1:100 dilution of the emulsion pre-concentrates of the present invention can be, e.g., between 0.3 and 4.0. For example, the absorbance at 400 nm can be about 0.4, 0.5, 0.6, 1.0, 1.2, 1.6, 2.0, 2.2, 2.4, 2.5, 3.0, or 4.0. Methods for determining the absorbance of a liquid solution are well known by those in the art. The skilled artisan will be able to ascertain and adjust the relative proportions of the ingredients of the emulsion pre-concentrates of the invention in order to obtain, upon dilution with water, an emulsion having any particular absorbance encompassed within the scope of the invention.

The pharmaceutical compositions of the present invention can be, e.g., in a solid, semi-solid, or liquid formulation. Semi-solid formulations of the present invention can be any semi-solid formulation known by those of ordinary skill in the art, including, e.g., gels, pastes, creams and ointments.

The pharmaceutical compositions of the present invention comprise a lipophilic phase component. Suitable components for use as lipophilic phase components include any pharmaceutically acceptable solvent which is non-miscible with water. Such solvents will appropriately be devoid or substantially devoid of surfactant function.

The lipophilic phase component may comprise mono-, di- or triglycerides. Mono-, di- and triglycerides that may be used within the scope of the invention include those that are derived from C₆, C₈, C₁₀, C₁₂, C₁₄, C₁₆, C₁₈, C₂₀ and C₂₂ fatty acids. Exemplary diglycerides include, in particular, diolein, dipalmitolein, and mixed caprylin-caprin diglycerides. Preferred triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

A preferred triglyceride is the medium chain triglyceride available under the trade name LABRAFAC CC. Other preferred triglycerides include neutral oils, e.g., neutral plant oils, in particular fractionated coconut oils such as known and commercially available under the trade name MIGLYOL, including the products: MIGLYOL 810; MIGLYOL 812; MIGLYOL 818; and CAPTEX 355.

Also suitable are caprylic-capric acid triglycerides such as known and commercially available under the trade name MYRITOL, including the product MYRITOL 813. Further suitable products of this class are CAPMUL MCT, CAPTEX 200, CAPTEX 300, CAPTEX 800, NEOBEE M5 and MAZOL 1400.

Especially preferred as lipophilic phase component is the product MIGLYOL 812. (See U.S. Pat. No. 5,342,625).

Pharmaceutical compositions of the present invention may further comprise a hydrophilic phase component. The hydrophilic phase component may comprise, e.g., a pharmaceutically acceptable C₁₋₅ alkyl or tetrahydrofurfuryl di- or partial-ether of a low molecular weight mono- or poly-oxy-alkanediol. Suitable hydrophilic phase components include, e.g., di -or partial-, especially partial-, -ethers of mono- or poly-, especially mono- or di-, -oxy-alkanediols comprising from 2 to 12, especially 4 carbon atoms. Preferably the mono- or poly-oxy-alkanediol moiety is straight-chained.

Exemplary hydrophilic phase components for use in relation to the present invention are those known and commercially available under the trade names TRANSCUTOL and COLYCOFUROL. (See U.S. Pat. No. 5,342,625).

In an especially preferred embodiment, the hydrophilic phase component comprises 1,2-propyleneglycol.

The hydrophilic phase component of the present invention may of course additionally include one or more additional ingredients. Preferably, however, any additional ingredients will comprise materials in which the active vitamin D compound is sufficiently soluble, such that the efficacy of the hydrophilic phase as an active vitamin D compound carrier medium is not materially impaired. Examples of possible additional hydrophilic phase components include lower (e.g., C₁₋₅) alkanols, in particular ethanol.

Pharmaceutical compositions of the present invention also comprise one or more surfactants. Surfactants that can be used in conjunction with the present invention include hydrophilic or lipophilic surfactants, or mixtures thereof. Especially preferred are non-ionic hydrophilic and non-ionic lipophilic surfactants.

Suitable hydrophilic surfactants include reaction products of natural or hydrogenated vegetable oils and ethylene glycol, i.e. polyoxyethylene glycolated natural or hydrogenated vegetable oils, for example polyoxyethylene glycolated natural or hydrogenated castor oils. Such products may be obtained in known manner, e.g., by reaction of a natural or hydrogenated castor oil or fractions thereof with ethylene oxide, e.g., in a molar ratio of from about 1:35 to about 1:60, with optional removal of free polyethyleneglycol components from the product, e.g., in accordance with the methods disclosed in German Auslegeschriften 1,182,388 and 1,518,819.

Suitable hydrophilic surfactants for use in the present pharmaceutical compounds also include polyoxyethylene-sorbitan-fatty acid esters, e.g., mono- and trilauryl, palmityl, stearyl and oleyl esters, e.g. of the type known and commercially available under the trade name TWEEN; including the products:

TWEEN 20 (polyoxyethylene(20)sorbitanmonolaurate),

TWEEN 40 (polyoxyethylene(20)sorbitanmonopalmitate),

TWEEN 60 (polyoxyethylene(20)sorbitanmonostearate),

TWEEN 80 (polyoxyethylene(20)sorbitanmonooleate),

TWEEN 65 (polyoxyethylene(20)sorbitantristearate),

TWEEN 85 (polyoxyethylene(20)sorbitantrioleate),

TWEEN 21 (polyoxyethylene(4)sorbitanmonolaurate),

TWEEN 61 (polyoxyethylene(4)sorbitanmonostearate), and

TWEEN 81 (polyoxyethylene(5)sorbitanmonooleate).

Especially preferred products of this class for use in the compositions of the invention are the above products TWEEN 40 and TWEEN 80. (See Hauer, et al., U.S. Pat. No. 5,342,625).

Also suitable as hydrophilic surfactants for use in the present pharmaceutical compounds are polyoxyethylene alkylethers; polyoxyethylene glycol fatty acid esters, for example polyoxythylene stearic acid esters;

polyglycerol fatty acid esters; polyoxyethylene glycerides; polyoxyethylene vegetable oils; polyoxyethylene hydrogenated vegetable oils; reaction mixtures of polyols and, e.g., fatty acids, glycerides, vegetable oils, hydrogenated vegetable oils, and sterols; polyoxyethylene-polyoxypropylene co-polymers; polyoxyethylene-polyoxypropylene block co-polymers; dioctylsuccinate, dioctylsodiumsulfosuccinate, di-[2-ethylhexyl]-succinate or sodium lauryl sulfate; phospholipids, in particular lecithins such as, e.g., soya bean lecithins; propylene glycol mono- and di-fatty acid esters such as, e.g., propylene glycol dicaprylate, propylene glycol dilaurate, propylene glycol hydroxystearate, propylene glycol isostearate, propylene glycol laurate, propylene glycol ricinoleate, propylene glycol stearate, and, especially preferred, propylene glycol caprylic-capric acid diester; and bile salts, e.g., alkali metal salts, for example sodium taurocholate.

Suitable lipophilic surfactants include alcohols; polyoxyethylene alkylethers; fatty acids; bile acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid esters of mono/diglycerides; propylene glycol diglycerides; sorbitan fatty acid esters; polyoxyethylene sorbitan fatty acid esters; polyoxyethylene-polyoxypropylene block copolymers; trans-esterified vegetable oils; sterols; sugar esters; sugar ethers; sucroglycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and mixtures thereof.

Suitable lipophilic surfactants for use in the present pharmaceutical compounds also include trans-esterification products of natural vegetable oil triglycerides and polyalkylene polyols. Such trans-esterification products are known in the art and may be obtained e.g., in accordance with the general procedures described in U.S. Pat. No. 3,288,824. They include trans-esterification products of various natural (e.g., non-hydrogenated) vegetable oils for example, maize oil, kernel oil, ahmond oil, ground nut oil, olive oil and palm oil and mixtures thereof with polyethylene glycols, in particular polyethylene glycols having an average molecular weight of from 200 to 800. Preferred are products obtained by trans-esterification of 2 molar parts of a natural vegetable oil triglyceride with one molar part of polyethylene glycol (e.g., having an average molecular weight of from 200 to 800). Various forms of trans-esterification products of the defined class are known and commercially available under the trade name LABRAFIL.

Additional lipophilic surfactants that are suitable for use with the present pharmaceutical compositions include oil-soluble vitamin derivatives, e.g., tocopherol PEG-1000 succinate (“vitamin E TPGS”).

Also suitable as lipophilic surfactants for use in the present pharmaceutical compounds are mono-, di- and mono/di-glycerides, especially esterification products of caprylic or capric acid with glycerol; sorbitan fatty acid esters; pentaerythritol fatty acid esters and polyalkylene glycol ethers, for example pentaerythrite- -dioleate, -distearate, -monolaurate, -polyglycol ether and -monostearate as well as pentaerythrite-fatty acid esters; monoglycerides, e.g., glycerol monooleate, glycerol monopalmitate and glycerol monostearate; glycerol triacetate or (1,2,3)-triacetin; and sterols and derivatives thereof, for example cholesterols and derivatives thereof, in particular phytosterols, e.g., products comprising sitosterol, campesterol or stigmasterol, and ethylene oxide adducts thereof, for example soya sterols and derivatives thereof.

It is understood by those of ordinary skill in the art that several commercial surfactant compositions contain small to moderate amounts of triglycerides, typically as a result of incomplete reaction of a triglyceride starting material in, for example, a trans-esterification reaction. Thus, the surfactants that are suitable for use in the present pharmaceutical compositions include those surfactants that contain a triglyceride. Examples of commercial surfactant compositions containing triglycerides include some members of the surfactant families GELUCIRES, MAISINES, and IMWITORS. Specific examples of these compounds are GELUCIRE 44/14 (saturated polyglycolized glycerides); GELUCIRE 50/13 (saturated polyglycolized glycerides);

GELUCIRE 53/10 (saturated polyglycolized glycerides); GELUCIRE 33/01 (semi-synthetic triglycerides of C₈-C₁₈ saturated fatty acids); GELUCIRE 39/01 (semi-synthetic glycerides); other GELUCIRES, such as 37/06, 43/01, 35/10, 37/02, 46/07, 48/09, 50/02, 62/05, etc.; MAISINE 35-I (linoleic glycerides); and IMWITOR OR 742 (caprylic/capric glycerides). (See U.S. Pat. No. 6,267,985).

Still other commercial surfactant compositions having significant triglyceride content are known to those skilled in the art. It should be appreciated that such compositions, which contain triglycerides as well as surfactants, may be suitable to provide all or part of the lipophilic phase component of the of the present invention, as well as all or part of the surfactants.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative of preferred or individually inventive teachings only and not as limiting the invention in its broadest aspect.

The lipophilic phase component of the invention will suitably be present in an amount of from about 30% to about 90% by weight based upon the total weight of the composition. Preferably, the lipophilic phase component is present in an amount of from about 50% to about 85% by weight based upon the total weight of the composition.

The surfactant or surfactants of the invention will suitably be present in an amount of from about 1% to 50% by weight based upon the total weight of the composition. Preferably, the surfactant(s) is present in an amount of from about 5% to about 40% by weight based upon the total weight of the composition.

The amount of active vitamin D compound in compositions of the invention will of course vary, e.g., depending on the intended route of administration and to what extent other components are present. In general, however, the active vitamin D compound of the invention will suitably be present in an amount of from about 0.005% to 20% by weight based upon the total weight of the composition. Preferably, the active vitamin D compound is present in an amount of from about 0.01% to 15% by weight based upon the total weight of the composition.

The hydrophilic phase component of the invention will suitably be present in an amount of from about 2% to about 20% by weight based upon the total weight of the composition. Preferably, the hydrophilic phase component is present in an amount of from about 5% to 15% by weight based upon the total weight of the composition.

The pharmaceutical composition of the invention may be in a semisolid formulation. Semisolid formulations within the scope of the invention may comprise, e.g., a lipophilic phase component present in an amount of from about 60% to about 80% by weight based upon the total weight of the composition, a surfactant present in an amount of from about 5% to about 35% by weight based upon the total weight of the composition, and an active vitamin D compound present in an amount of from about 0.01% to about 15% by weight based upon the total weight of the composition.

The pharmaceutical compositions of the invention may be in a liquid formulation. Liquid formulations within the scope of the invention may comprise, e.g., a lipophilic phase component present in an amount of from about 50% to about 60% by weight based upon the total weight of the composition, a surfactant present in an amount of from about 4% to about 25% by weight based upon the total weight of the composition, an active vitamin D compound present in an amount of from about 0.01% to about 15% by weight based upon the total weight of the composition, and a hydrophilic phase component present in an amount of from about 5% to about 10% by weight based upon the total weight of the composition.

Additional compositions that may be used include the following, wherein the percentage of each component is by weight based upon the total weight of the composition excluding the active vitamin D compound:

| | | |
|---|---|---|
| a. | Gelucire 44/14 | about 50% |
| | Miglyol 812 | about 50%; |
| b. | Gelucire 44/14 | about 50% |
| | Vitamin E TPGS | about 10% |
| | Miglyol 812 | about 40%; |
| c. | Gelucire 44/14 | about 50% |
| | Vitamin E TPGS | about 20% |
| | Miglyol 812 | about 30%; |
| d. | Gelucire 44/14 | about 40% |
| | Vitamin E TPGS | about 30% |
| | Miglyol 812 | about 30%; |
| e. | Gelucire 44/14 | about 40% |
| | Vitamin E TPGS | about 20% |
| | Miglyol 812 | about 40%; |
| f. | Gelucire 44/14 | about 30% |
| | Vitamin E TPGS | about 30% |
| | Miglyol 812 | about 40%; |
| g. | Gelucire 44/14 | about 20% |
| | Vitamin E TPGS | about 30% |
| | Miglyol 812 | about 50%; |
| h. | Vitamin E TPGS | about 50% |
| | Miglyol 812 | about 50%; |
| i. | Gelucire 44/14 | about 60% |
| | Vitamin E TPGS | about 25% |
| | Miglyol 812 | about 15%; |
| j. | Gelucire 50/13 | about 30% |
| | Vitamin E TPGS | about 5% |
| | Miglyol 812 | about 65%; |
| k. | Gelucire 50/13 | about 50% |
| | Miglyol 812 | about 50%; |
| l. | Gelucire 50/13 | about 50% |
| | Vitamin E TPGS | about 10% |
| | Miglyol 812 | about 40%; |
| m. | Gelucire 50/13 | about 50% |
| | Vitamin E TPGS | about 20% |
| | Miglyol 812 | about 30%; |
| n. | Gelucire 50/13 | about 40% |
| | Vitamin E TPGS | about 30% |
| | Miglyol 812 | about 30%; |
| o. | Gelucire 50/13 | about 40% |
| | Vitamin E TPGS | about 20% |
| | Miglyol 812 | about 40%; |
| p. | Gelucire 50/13 | about 30% |
| | Vitamin E TPGS | about 30% |
| | Miglyol 812 | about 40%; |
| q. | Gelucire 50/13 | about 20% |
| | Vitamin E TPGS | about 30% |
| | Miglyol 812 | about 50%; |
| r. | Gelucire 50/13 | about 60% |
| | Vitamin E TPGS | about 25% |
| | Miglyol 812 | about 15%; |
| s. | Gelucire 44/14 | about 50% |
| | PEG 4000 | about 50%; |
| t. | Gelucire 50/13 | about 50% |
| | PEG 4000 | about 50%; |
| u. | Vitamin E TPGS | about 50% |
| | PEG 4000 | about 50%; |
| v. | Gelucire 44/14 | about 33.3% |
| | Vitamin E TPGS | about 33.3% |
| | PEG 4000 | about 33.3%; |
| w. | Gelucire 50/13 | about 33.3% |
| | Vitamin E TPGS | about 33.3% |
| | PEG 4000 | about 33.3%; |
| x. | Gelucire 44/14 | about 50% |
| | Vitamin E TPGS | about 50%; |
| y. | Gelucire 50/13 | about 50% |
| | Vitamin E TPGS | about 50%; |
| z. | Vitamin E TPGS | about 5% |
| | Miglyol 812 | about 95%; |
| aa. | Vitamin E TPGS | about 5% |
| | Miglyol 812 | about 65% |
| | PEG 4000 | about 30%; |
| ab. | Vitamin E TPGS | about 10% |
| | Miglyol 812 | about 90%; |
| ac. | Vitamin E TPGS | about 5% |
| | Miglyol 812 | about 85% |
| | PEG 4000 | about 10%; and |
| ad. | Vitamin E TPGS | about 10% |
| | Miglyol 812 | about 80% |
| | PEG 4000 | about 10%. |

In one embodiment of the invention, the pharmaceutical compositions comprise an active vitamin D compound, a lipophilic component, and a surfactant. The lipophilic component may be present in any percentage from about 1% to about 100%. The lipophilic component may be present at about 1, 2, 3, 4, 5, 6, 7, 8, 9, 10, 11, 12, 13, 14, 15, 16, 17, 18, 19, 20, 21,22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant maybe present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In preferred embodiments, the pharmaceutical compositions comprise 50% MIGLYOL 812 and 50% vitamin E TPGS, 90% MIGLYOL 812 and 10% vitamin E TPGS, or 95% MIGLYOL 812 and 5% vitamin E TPGS.

In another embodiment of the invention, the pharmaceutical compositions comprise an active vitamin D compound and a lipophilic component, e.g., around 100% MIGLYOL 812.

In a preferred embodiment, the pharmaceutical compositions comprise 50% MIGLYOL 812, 50% vitamin E TPGS, and small amounts of BHA and BHT. This formulation has been shown to be unexpectedly stable, both chemically and physically (see Example 3). The enhanced stability provides the compositions with a longer shelf life. Importantly, the stability also allows the compositions to be stored at room temperature, thereby avoiding the complication and cost of storage under refrigeration. Additionally, this composition is suitable for oral administration and has been shown to be capable of solubilizing high doses of active vitamin D compound, thereby enabling high dose pulse administration of active vitamin D compounds for the treatment of hyperproliferative diseases and other disorders.

The pharmaceutical compositions comprising the active vitamin D compound of the present invention may further comprise one or more additives. Additives that are well known in the art include, e.g., detackifiers, anti-foaming agents, buffering agents, antioxidants (e.g., ascorbyl palmitate, butyl hydroxy anisole (BHA), butyl hydroxy toluene (BHT) and tocopherols, e.g., α-tocopherol (vitamin E)), preservatives, chelating agents, viscomodulators, tonicifiers, flavorants, colorants odorants, opacifiers, suspending agents, binders, fillers, plasticizers, lubricants, and mixtures thereof. The amounts of such additives can be readily determined by one skilled in the art, according to the particular properties desired. For example, antioxidants may be present in an amount of from about 0.05% to about 0.35% by weight based upon the total weight of the composition.

The additive may also comprise a thickening agent. Suitable thickening agents may be those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g. hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents as aforesaid will generally not be required and is generally less preferred. Use of thickening agents is, on the other hand, indicated, e.g., where topical application is foreseen.

Compositions in accordance with the present invention may be employed for administration in any appropriate manner, e.g., orally, e.g. in unit dosage form, for example in a solution, in hard or soft encapsulated form including gelatin encapsulated form, parenterally or topically, e.g., for application to the skin, for example in the form of a cream, paste, lotion, gel, ointment, poultice, cataplasm, plaster, dermal patch or the like, as a coating for a medical device, e.g., a stent, or for ophthalmic application, for example in the form of an eye-drop, -lotion or -gel formulation. Readily flowable forms, for example solutions and emulsions, may also be employed e.g., for intralesional injection, or may be administered rectally, e.g., as an enema.

When the composition of the present invention is formulated in unit dosage form, the active vitamin D compound will preferably be present in an amount of between 1 and 200 μg per unit dose. More preferably, the amount of active vitamin D compound per unit dose will be about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 μg or any amount therein. In a preferred embodiment, the amount of active vitamin D compound per unit dose will be about 5 μg to about 180 μg, more preferably about 10 μg to about 135 μg, more preferably about 45 μg. In one embodiment, the unit dosage form comprises 45, 90, 135, or 180 μg of calcitriol.

When the unit dosage form of the composition is a capsule, the total quantity of ingredients present in the capsule is preferably about 10-1000 μL.

More preferably, the total quantity of ingredients present in the capsule is about 100-300 μL. In another embodiment, the total quantity of ingredients present in the capsule is preferably about 10-1500 mg, preferably about 100-1000 mg. In one embodiment, the total quantity is about 225, 450, 675, or 900 mg. In one embodiment, the unit dosage form is a capsule comprising 45, 90, 135, or 180 μg of calcitriol.

Animals which may be treated according to the present invention include all animals which may benefit from administration of the compounds-of the present invention. Such animals include humans, pets such as dogs and cats, and veterinary animals such as cows, pigs, sheep, goats and the like.

The following examples are illustrative, but not limiting, of the methods of the present invention. Other suitable modifications and adaptations of the variety of conditions and parameters normally encountered in medical treatment and pharmaceutical science and which are obvious to those skilled in the art are within the spirit and scope of the invention.

### EXAMPLE 1

### Preperation of Semi-Solid Calcitriol Formulations

Five semi-solid calcitriol formulations (SS1-SS5) were prepared containing the ingredients listed in Table 1. The final formulation contains 0.208 mg calcitriol per gram of semi-solid formulation.

TABLE 1
Composition of Semi-Solid Calcitriol Formulation

| Ingredients | SS1 | SS2 | SS3 | SS4 | SS5 |
|---|---|---|---|---|---|
| Calcitriol | 0.0208 | 0.0208 | 0.0208 | 0.0208 | 0.0208 |
| Miglyol 812 | 80.0 | 0 | 65.0 | 0 | 79.0 |
| Captex 200 | 0 | 82.0 | 0 | 60.0 | 0 |
| Labrafac CC | 0 | 0 | 0 | 0 | 12.0 |
| Vitamin-E TPGS | 20.0 | 18.0 | 5.0 | 5.0 | 9.0 |
| Labrifil M | 0 | 0 | 0 | 0 | 0 |
| Gelucire 44/14 | 0 | 0 | 30.0 | 35.0 | 0 |
| BHT | 0.05 | 0.05 | 0.05 | 0.05 | 0.05 |
| BHA | 0.05 | 0.05 | 0.05 | 0.05 | 0.05 |

Amounts shown are in grams.

1. Preparation of Vehicles

One hundred gram quantities of the five semi-solid calcitriol formulations (SS1-SS5) listed in Table 1 were prepared as follows.

The listed ingredients, except for calcitriol, were combined in a suitable glass container and mixed until homogenous. Vitamin E TPGS and GELUCIRE 44/14 were heated and homogenized at 60° C. prior to weighing and adding into the formulation.

2. Preparation of Active Formulations

The semi-solid vihicles were heated and homogenized at ≦60° C. Under subdued light, 12≦1 mg of calcitriol was weighed out into separate glass bottles with screw caps, one bottle for each formulation. (Calcitriol is light sensitive; subdued light/red light should be used when working with calcitriol/calcitriol formulations.) The exact weight was recorded to 0.1 mg. The caps were then placed on the bottles as soon as the calcitriol had been placed into the bottles. Next, the amount of each vehicle required to bring the concentration to 0.208 mg/g was calculated using the following formula:
C_{W}/0.208=required weight of vehicle
Where C_{W}=weight of calcitriol, in mg, and
0.1208=final concentration of calcitriol (mg/g).

Finally, the appropriate amount of each vehicle was added to the respective bottle containing the calcitriol. The formulations were heated (≦60° C.) while being mixed to dissolve the calcitriol.

### EXAMPLE 2

### Preparation of Additional Formalations

Following the method of Example 1, twelve different formulations for calcitriol were prepared containing the ingredients listed in Table 2.

TABLE 2
Composition Formulations

| Ingre- | | | | | | | | | | | | |
|---|---|---|---|---|---|---|---|---|---|---|---|---|
| dients | 1 | 2 | 3 | 4 | 5 | 6 | 7 | 8 | 9 | 10 | 11 | 12 |
| Miglyol | 95 | 65 | 90 | 85 | 80 | 95 | 65 | 90 | 85 | 80 | 50 | 0 |
| 812N | | | | | | | | | | | | |
| Vitamin | 5 | 5 | 10 | 5 | 10 | 5 | 5 | 10 | 5 | 10 | 50 | 50 |
| E TPGS | | | | | | | | | | | | |
| PEG | 0 | 30 | 0 | 10 | 10 | 0 | 30 | 0 | 10 | 10 | 0 | 50 |
| 4000 | | | | | | | | | | | | |
| BHA | 0.05 | 0.05 | 0.05 | 0.05 | 0.05 | 0.35 | 0.35 | 0.35 | 0.35 | 0.35 | 0.35 | 0.35 |
| BHT | 0.05 | 0.05 | 0.05 | 0.05 | 0.05 | 0.35 | 0.35 | 0.35 | 0.35 | 0.35 | 0.35 | 0.35 |

Amounts shown are percentages.

### EXAMPLE 3

### Stable Unit Dose Formulations

Formulations of calcitriol were prepared to yield the compositions in Table 3. The Vitamin E TPGS was warmed to approximately 50° C. and mixed in the appropriate ratio with MIGLYOL 812. BHA and BHT were added to each formulation to achieve 0.35% w/w of each in the final preparations.

TABLE 3
Calcitriol formulations

| | MIGLYOL | Vitamin E TPGS |
|---|---|---|
| Formulation # | (% wt/wt) | (% wt/wt) |
| 1 | 100 | 0 |
| 2 | 95 | 5 |
| 3 | 90 | 10 |
| 4 | 50 | 50 |

After formulation preparation, Formulations 2-4 were heated to approximately 50° C. and mixed with calcitriol to produce 0.1 μg calcitriol/mg total formulation. The formulations contained calcitriol were then added (˜250 μL) to a 25 mL volumetric flask and deionized water was added to the 25 mL mark. The solutions were then vortexed and the absorbance of each formulation was measured at 400 nm immediately after mixing (initial) and up to 10 min after mixing. As shown in Table 4, all three formulations produced an opalescent solution upon mixing with water. Formulation 4 appeared to form a stable suspension with no observable change in absorbance at 400 nm after 10 min.

TABLE 4
Absorption of formulations suspended in water

| | Absorbance at 400 nm | |
|---|---|---|
| Formulation # | Initial | 10 min |
| 2 | 0.7705 | 0.6010 |
| 3 | 1.2312 | 1.1560 |
| 4 | 3.1265 | 3.1265 |

To further assess the formulations of calcitriol, a solubility study was conducted to evaluate the amount of calcitriol soluble in each formulation. Calcitriol concentrations from 0.1 to 0.6 μg calcitriol/mg formulation were prepared by heating the formulations to 50° C. followed by addition of the appropriate mass of calcitriol. The formulations were then allowed to cool to room temperature and the presence of undissolved calcitriol was determined by a light microscope with and without polarizing light. For each formulation, calcitriol was soluble at the highest concentration tested, 0.6 μg calcitriol/mg formulation.

A 45 μg calcitriol dose is currently being used in Phase 2 human clinical trials. To develop a capsule with this dosage each formulation was prepared with 0.2 μg calcitriol/mg formulation and 0.35% w/w of both BHA and BHT. The bulk formulation mixtures were filled into Size 3 hard gelatin capsules at a mass of 225 mg (45 μg calcitriol). The capsules were then analyzed for stability at 5° C., 25° C./60% relative humidity (RH), 30° C./65% RH, and 40° C./75% RH. At the appropriate time points, the stability samples were analyzed for content of intact calcitriol and dissolution of the capsules. The calcitriol content of the capsules was determined by dissolving three opened capsules in 5 mL of methanol and held at 5° C. prior to analysis. The dissolved samples were then analyzed by reversed phase HPLC. A Phemonex Hypersil BDS C18 column at 30° C. was used with a gradient of acetonitrile from 55% acetonitrile in water to 95% acetonitrile at a flow rate of 1.0 mL/min during elution. Peaks were detected at 265 nm and a 25 μL sample was injected for each run. The peak area of the sample was compared to a reference standard to calculate the calcitriol content as reported in Table 5. The dissolution test was performed by placing one capsule in each of six low volume dissolution containers with 50 mL of deionized water containing 0.5% sodium dodecyl sulfate. Samples were taken at 30, 60 and 90 min after mixing at 75 rpm and 37° C. Calcitriol content of the samples was determined by injection of 100 μL samples onto a Betasil C18 column operated at 1 mL/min with a mobile phase of 50:40:10 acetonitrile:water:tetrahydrofuran at 30° C. (peak detection at 265 nm). The mean value from the 90 min dissolution test results of the six capsules was reported (Table 6).

TABLE 5
Chemical stability of calcitriol formulation in hard gelatin capsules
(225 mg total mass filled per capsule, 45 μg calcitriol)

| Storage | Time | Assay^{a} (%) | | | |
|---|---|---|---|---|---|
| Condition | (mos) | Form. 1 | Form. 2 | Form 3 | Form 4 |
| N/A | 0 | 100.1 | 98.8 | 99.1 | 100.3 |
| 5° C. | 1.0 | 99.4 | 98.9 | 98.9 | 104.3 |
| 25° C./60% RH | 0.5 | 99.4 | 97.7 | 97.8 | 102.3 |
| | 1.0 | 97.1 | 95.8 | 97.8 | 100.3 |
| | 3.0 | 95.2 | 93.6 | 96.8 | 97.9 |
| 30° C./65% RH | 0.5 | 98.7 | 97.7 | 96.8 | 100.7 |
| | 1.0 | 95.8 | 96.3 | 97.3 | 100.4 |
| | 3.0 | 94.2 | 93.6 | 95.5 | 93.4 |
| 40° C./75% RH | 0.5 | 96.4 | 96.7 | 98.2 | 97.1 |
| | 1.0 | 96.1 | 98.6 | 98.5 | 99.3 |
| | 3.0 | 92.3 | 92.4 | 93.0 | 96.4 |

^{a}Assay results indicate % of calcitriol relative to expected value based upon 45 μg content per capsule. Values include pre-calcitriol which is an active isomer of calcitriol.

TABLE 6
Physical Stability of Calcitriol Formulation in Hard Gelatin
Capsules (225 mg total mass filled per capsule, 45 μg calcitriol)

| Storage | Time | Dissolution^{a} (%) | | | |
|---|---|---|---|---|---|
| Condition | (mos) | Form. 1 | Form. 2 | Form 3 | Form 4 |
| N/A | 0 | 70.5 | 93.9 | 92.1 | 100.1 |
| 5° C. | 1.0 | 71.0 | 92.3 | 96.0 | 100.4 |
| 25° C./60% RH | 0.5 | 65.0 | 89.0 | 90.1 | 98.3 |
| | 1.0 | 66.1 | 90.8 | 94.5 | 96.2 |
| | 3.0 | 64.3 | 85.5 | 90.0 | 91.4 |
| 30° C./65% RH | 0.5 | 62.1 | 88.8 | 91.5 | 97.9 |
| | 1.0 | 65.1 | 89.4 | 95.5 | 98.1 |
| | 3.0 | 57.7 | 86.4 | 89.5 | 88.8 |
| 40° C./75% RH | 0.5 | 91.9 | 90.2 | 92.9 | 93.1 |
| | 1.0 | 63.4 | 93.8 | 94.5 | 95.2 |
| | 3.0 | 59.3 | 83.6 | 87.4 | 91.1 |

^{a}Dissolution of capsules was performed as described and the % calcitriol is calculated based upon a standard and the expected content of 45 μg calcitriol per capsule. The active isomer, pre-calcitriol, is not included in the calculation of % calcitriol dissolved. Values reported are from the 90 min sample.

The chemical stability results indicated that decreasing the MIGLYOL 812 content with a concomitant increase in Vitamin E TPGS content provided enhanced recovery of intact calcitriol as noted in Table 5. Formulation 4 (50:50 MGLYOL 812/Vitamin E TPGS) was the most chemically stable formulation with only minor decreases in recovery of intact calcitriol after 3 months at 25° C./60% RH, enabling room temperature storage.

The physical stability of the formulations was assessed by the dissolution behavior of the capsules after storage at each stability condition. As with the chemical stability, decreasing the MIGLYOL 812 content and increasing the Vitamin E TPGS content improved the dissolution properties of the formulation (Table 6). Formulation 4 (50:50 MIGLYOL 812/Vitamin E TPGS) had the best dissolution properties with suitable stability for room temperature storage.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1-42. (canceled)

43. A method for preventing, treating, or ameliorating restenosis after angioplasty in an animal, comprising administering to the animal a therapeutically effective amount of an active vitamin D compound coated or impregnated in a stent placed at the site of the angioplasty.

44. The method of claim 43, wherein the active vitamin D compound is contained within a matrix which is coated on or impregnated in the stent.

45. The method of claim 44, wherein the matrix controls the release of the active vitamin D compound.

46. The method of any one of claims 43-45, wherein the active vitamin D compound is calcitriol.
